(19) United States
(12) Patent Application Publication
(10) Pub. No.: US 2016/0128861 A1
(43) Pub. Date: May 12, 2016

(54) Garment with integral orthopedic support structure

(71) Applicant: Kimberly L. Plaza, Houston, TX (US)

(72) Inventor: Kimberly L. Plaza, Houston, TX (US)

(21) Appl. No.: 13/987,454

(22) Filed: Jul. 29, 2013

(51) Int. Cl. A61F 5/02; A41B 1/08

(52) U.S. Cl. CPC A61F 5/028; A41B 1/08

(57) ABSTRACT

An orthopedic support apparatus is provided in the form of a substantially elongate support element sized and configured to provide back support to the wearer. In one embodiment, the support element is affixed inside the back of a garment, such as a shirt. Belt flaps are provided to encircle the wearer's torso and maintain the support element against the user's back. The belt flaps are connected only to the base of the support element and not to the garment, such that the garment can be worn without the support element being noticeable.

### FIELD OF THE INVENTION

The present invention relates generally to orthopedic support devices, and more particularly relates to a garment having an integral orthopedic support structure.

### BACKGROUND OF THE INVENTION

There are numerous examples of orthopedic support apparatuses in the prior art. Such apparatuses are provided to provide orthopedic support for persons with poor posture or who are regularly engaged in activities which involve orthopedic stress (e.g., heavy lifting). Examples in the prior art include the following:

U.S. Pat. No. 8,087,094 to Karasina, entitled “Shirt Having Form-Fitting Mid-Section Support;” U.S. Pat. No. 8,047,893 to Fenske, entitled “Posture Support Garment;” U.S. Pat. No. 6,766,532 to Cabana, entitled “Adjustable Back Support Belt Brace System and Removable, Adjustable Suspension System;” U.S. Pat. No. 6,314,580 to Greenberg et al., entitled “Upper Body Support Jacket;” U.S. Pat. No. 6,205,591 to Wheeler et al., entitled “Tailored Garment With Integral Support Unit;” U.S. Pat. No. 6,315,747 to Toole, entitled “Posture Support Apparatus;” U.S. Pat. No. 6,238,266 to Vogt, entitled “Support Garment Fabrics with Cross-Linked Polyurethane Latices Coated Thereon;” U.S. Pat. No. 7,134,969 to Citron et al., entitled “Golf Posture Brace and Garment;” and U.S. Pat. No. 6,440,094 to Maas, entitled “Orthopedic Garment for Dynamically Enhancing Proper Posture.”

One aspect of orthopedic apparatuses in the prior art that does not seem to have been adequately addressed is the ability to provide orthopedic support in a manner that is aesthetically unobtrusive, such that the apparatus can be utilized in daily activities without causing undue attention or embarrassment to the user.

### SUMMARY OF THE INVENTION

In view of the foregoing and other considerations, the present invention is directed to an orthopedic apparatus for provide support for a wearer. In accordance with one aspect of the invention, a substantially elongate support element is provided which is sized and positioned to provide support generally along the back and spine of the wearer.

In accordance with another aspect of the invention, the support element is affixed inside a garment, such as a shirt. There are provided belt flaps for maintaining the support element adjacent to the wearer's back. In one embodiment, the belt flaps are secured to one another with Velcro® tabs and encircle the wearer's waist/torso.

In accordance with another aspect of the invention, the belt flaps are not attached to the garment, such that when the garment is worn by the wearer, the presence of the support element is substantially undetectable.

### BRIEF DESCRIPTION OF THE DRAWINGS

The present invention is best understood with reference to the following detailed description of embodiments of the invention when read in conjunction with the attached drawing, in which:

FIG. 1 is a frontal view of an orthopedic support apparatus in accordance with one embodiment of the invention.

### DETAILED DESCRIPTION OF A PREFERRED EMBODIMENT OF THE INVENTION

In the disclosure that follows, in the interest of clarity, not all features of actual implementations are described. It will of course be appreciated that in the development of any such actual implementation, as in any such project, numerous engineering and technical decisions must be made to achieve the developers' specific goals and subgoals (e.g., compliance with system and technical constraints), which will vary from one implementation to another. Moreover, attention will necessarily be paid to proper engineering practices for the environment in question. It will be appreciated that such development efforts might be complex and time-consuming, outside the knowledge base of typical laymen, but would nevertheless be a routine undertaking for those of ordinary skill in the relevant fields.

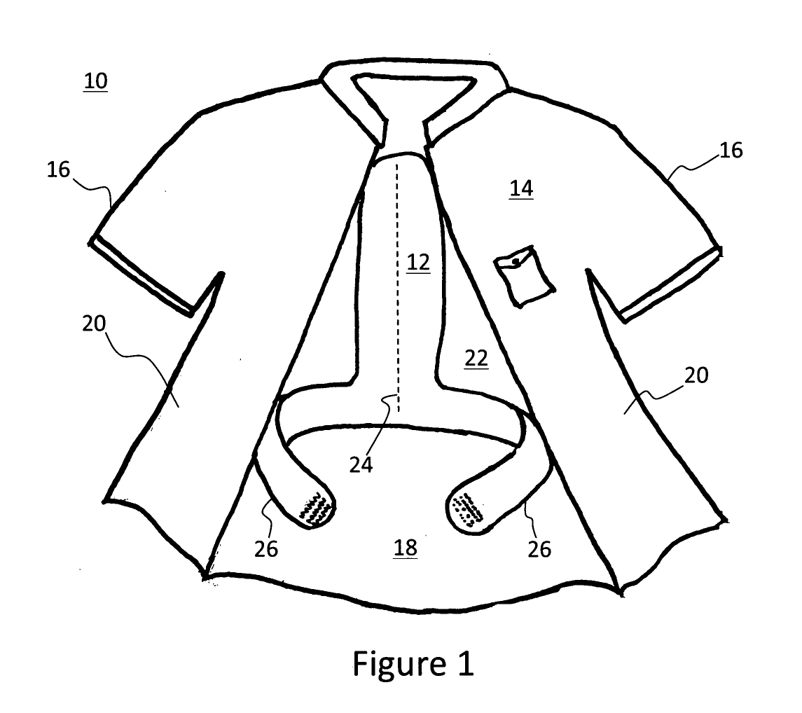

Referring to FIG. 1, there is shown a frontal view of an orthopedic support apparatus 10 in accordance with one embodiment of the invention. The support apparatus includes an elongate support member 12 which is sized and adapted to provide orthopedic support to a wearer, primarily by extending substantially along the spine of the wearer from the lower back towards the neck.

In a preferred embodiment, support member 12 is substantially rigid, being made of plastic, metal, or other suitable materials. In one embodiment, support member 12 is provided with padding along its length.

As shown in FIG. 1, apparatus 10 also comprises a garment 14, which in the presently disclosed embodiment comprises a shirt having sleeves 16 and a body portion 18 including left and right front portions 20.

In accordance with one aspect of the invention, elongate support member 12 is affixed to a back portion 22 of garment 14, preferably at one or more locations along the axial length of support member 12 as represented by dashed line 24

At least one embodiment of the invention has been described herein solely for the purposes of illustrating the invention in its various aspects. It is contemplated and to be explicitly understood that various substitutions, alterations, and/or modifications, including but not limited to any such implementation variants and options as may have been specifically noted or suggested herein, including inclusion of technological enhancements to any particular method step or system component discovered or developed subsequent to the date of this disclosure, may be made to the disclosed embodiments of the invention without necessarily departing from the technical and legal scope of the invention as defined in the following claims.

What is claimed is:

1. An apparatus for providing orthopedic support for a wearer, comprising:
an elongate support member sized and adapted to extend along at least a portion of said wearer's spine; and
opposing first and second belt flaps coupled substantially near a lower portion of said support member and adapted to encircle said wearer's torso to maintain substantial contact between said support member and said wearer's spine;
wherein said elongate support member is secured inside a garment having a body portion and sleeves, such that the garment substantially conceals said support member and said first and second belt flaps.

2. An apparatus in accordance with claim 1, wherein said first and second belt flaps engage one another at respective distal ends thereof.

3. An apparatus in accordance with claim 2, wherein said first and second belt flaps have Velcro® tabs at said respective distal ends.

4. An apparatus in accordance with claim 1, wherein said garment comprises a shirt.

5. An apparatus in accordance with claim 4, wherein said garment opens in the front.

6. A method of providing orthopedic support for a wearer, comprising:
providing an elongate support member sized and adapted to extend along at least a portion of said wearer's spine; and
providing opposing first and second belt flaps coupled substantially near a lower portion of said support member and adapted to encircle said wearer's torso to maintain substantial contact between said support member and said wearer's spine;
wherein said elongate support member is secured inside a garment having a body portion and sleeves, such that the garment substantially conceals said support member and said first and second belt flaps.

7. A method in accordance with claim 6, wherein said first and second belt flaps engage one another at respective distal ends thereof.

8. A method in accordance with claim 7, wherein said first and second belt flaps have Velcro® tabs at said respective distal ends.

9. A method in accordance with claim 6, wherein said garment comprises a shirt.

10. A method in accordance with claim 9, wherein said garment opens in the front.
